(19) United States
(12) Patent Application Publication
(10) Pub. No.: US 2008/0140451 A1
(43) Pub. Date: Jun. 12, 2008

(54) Devices and Methods for Monitoring, Managing, and Servicing Medical Devices

(75) Inventors: Marc H. Hedrick, Encinitas, CA (US); Christopher Calhoun, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: CYTORI THERAPEUTICS, INC.

(21) Appl. No.: 11/813,579

(22) PCT Filed: Jan. 12, 2005
(86) PCT No.: PCT/US05/01267

§ 371 Date: Dec. 13, 2007

Related U.S. Application Data
(60) Provisional application No. 60/642,954, filed on Jan. 10, 2005.

(51) Int. Cl. G06F 19/00; C12N 5/08

(52) U.S. Cl. 705/3; 424/93.7

(57) ABSTRACT

A method and apparatus for billing entities for use of a medical device, which relates to the processing of regenerative cells to facilitate at least one of hard and soft tissue formation, are described. When an operator uses the medical device, a corresponding charge is determined. Billing for usage of the medical device is performed on a periodic or per-use basis. Communication over a network facilitates remote management and servicing of the medical device. Support is provided for automatic ordering of supplies consumed by the medical device.

### CROSS-REFERENCE TO RELATED APPLICATIONS

This application claims the benefit of U.S. Provisional Application No. ______, filed Jan. 10, 2005 and entitled DEVICES AND METHODS FOR INDUCING TISSUE FORMATION AND BILLING A USER OF THE DEVICES AND METHODS. This application further is a continuation-in-part application of U.S. application Ser. No. 10/884,638, filed Jul. 2, 2004 and entitled “SYSTEMS AND METHODS FOR ISOLATING AND USING CLINICALLY SAFE ADIPOSE DERIVED REGENERATIVE CELLS,” which is a continuation-in-part of U.S. application Ser. No. 10/316,127, filed Dec. 9, 2002 and entitled “SYSTEMS AND METHODS FOR TREATING PATIENTS WITH PROCESSED LIPOASPIRATE CELLS,” which claims the benefit of U.S. Provisional Application No. 60/338,856, filed Dec. 7, 2001. The contents of all the aforementioned applications are expressly incorporated herein in their entirety by reference.

### BACKGROUND OF THE INVENTION

1. Field of the Invention

The present invention relates generally to methods and apparatus for monitoring medical devices, e.g., charging for use of medical devices, as well as methods and apparatus for managing and servicing medical devices, e.g., remotely managing and servicing medical devices. More particularly, the present invention relates to providing methods and apparatus for charging for use of a medical device for separating and concentrating clinically safe regenerative cells from adipose tissue.

2. Description of Related Art

As can be understood from the description of related art presented in the aforementioned applications, which are herein incorporated by reference, a need exists for apparatus, devices, systems and methods for extracting regenerative cells from adipose tissues in a matter suitable for direct placement into a recipient. Because application of such apparatus, devices, systems and methods to practical problem solving related to monitoring, managing and servicing incurs an associated cost, an additional need exists for methods and apparatus capable of aiding in the recovery of such costs.

### SUMMARY OF THE INVENTION

The present invention addresses the need for monitoring usage of a medical device, such as a medical device capable of inducing hard or soft tissue formation, by providing a medical device capable of inducing hard or soft tissue formation, wherein the medical device causes a user to be billed for use of the medical device. An embodiment of the invention herein disclosed comprises a concentrator capable of concentrating regenerative cells from tissue to form a composition capable of being placed into a patient to induce at least one of hard and soft tissue formation.

Another embodiment of the present invention comprises a medical billing device comprising a storage device, a printer, and a processor. The processor in this embodiment may be programmed to receive a usage event and to print on the printer a report describing a user charge according to the usage event.

Another embodiment of the present invention comprises a method of billing a user of at least one medical device. An implementation of this method comprises maintaining a database of user charge codes and usage events according to the at least one medical device and receiving a usage event and a user charge code. A billed amount then may be determined according to the usage event and the user charge code, and a report may be generated according to the billed amount. In other embodiments, the present invention comprises methods and apparatus for managing and servicing the medical devices. In certain embodiments, the medical devices can be managed and/or serviced remotely. Such embodiments can comprise a medical device such as the medical devices capable of inducing hard or soft tissue formation that is connected to a wired or wireless network. Methods, apparatus and advantages of such embodiments are further described herein.

While the apparatus and method has or will be described for the sake of grammatical fluidity with functional explanations, it is to be expressly understood that the claims, unless expressly formulated under 35 U.S.C. 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. 112 are to be accorded full statutory equivalents under 35 U.S.C. 112.

Any feature or combination of features described herein are included within the scope of the present invention provided that the features included in any such combination are not mutually inconsistent as will be apparent from the context, this specification, and the knowledge of one skilled in the art. For purposes of summarizing the present invention, certain aspects, advantages and novel features of the present invention are described or incorporated by reference herein. Of course, it is to be understood that not necessarily all such aspects, advantages or features will be embodied in any particular embodiment of the present invention. Additional advantages and aspects of the present invention are apparent in the following detailed description and claims that follow.

### BRIEF DESCRIPTION OF THE DRAWINGS

FIG. 1 is a partial block diagram of a medical device capable of inducing hard or soft tissue formation according to the present invention;

FIG. 2 is a block diagram of a programmable processing device;

FIG. 3 is a pictorial diagram depicting a medical billing device;

FIG. 4 is a chart illustrating a data structure for storing charge codes and rates;

FIG. 5 is a chart describing a data structure for storing record of medical device usage events;

FIG. 6 is a chart depicting a report comprising a bill according to a single use of a medical device;

FIG. 7 is a chart illustrating a report comprising a periodic billing for uses of medical devices;

FIG. 8 is a flow diagram describing an implementation of a method of billing a user for use of one or more medical devices;

FIG. 9 is a table listing instruction sequences included in an exemplary medical billing device;

FIG. 10 is a flow diagram describing an implementation of a method for creating a report according to usage data for one or more medical devices;

FIG. 11 is a flow diagram depicting an example of automated inventory management, ordering, and billing according to the present invention;

FIG. 12 is a table comprising a usage report generated according to the present invention; and

FIG. 13 is a chart illustrating an example of a usage report presented in graphical form.

### DETAILED DESCRIPTION OF THE INVENTION

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. It is to be understood and appreciated that the process steps and structures described or incorporated by reference herein do not cover a complete process flow for the implementation of medical billing devices. The present invention may be practiced in conjunction with various medical devices that are conventionally used in the art, and only so much of the commonly practiced process steps are included herein as are necessary to provide an understanding of the present invention. The present invention has applicability in the field of medical devices and processes in general. For illustrative purposes, however, the following description pertains to a medical device capable of isolating and using clinically safe adipose derived regenerative cells and to a method of billing for use of the medical device.

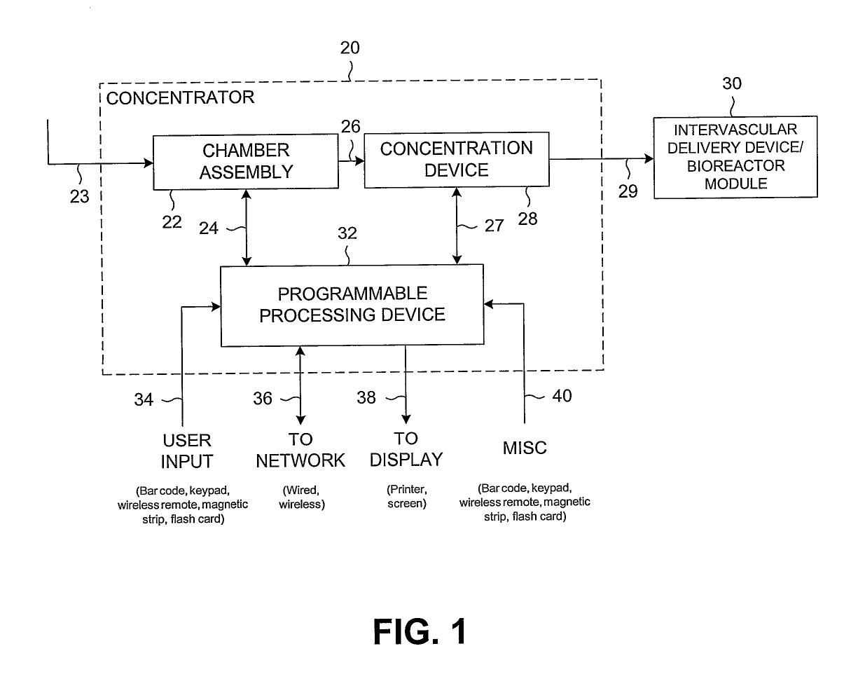

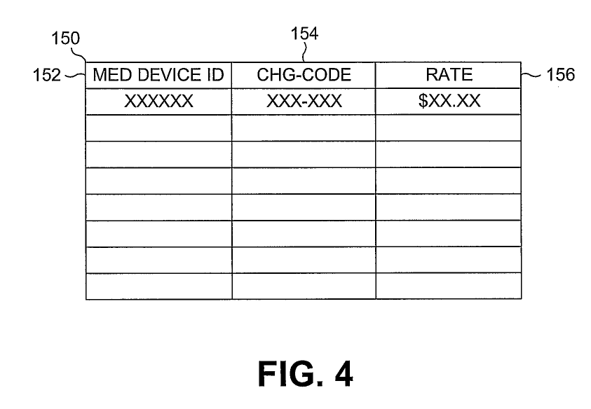

Referring more particularly to the drawings, FIG. 1 is a partial block diagram of an example of a medical device capable of inducing hard or soft tissue formation according to the present invention. Elements of the invention are grouped in FIG. 1 so as define a system (e.g., a partially or fully automated system) or concentrator 20 comprising a programmable processing device 32 (e.g., a microprocessor or personal computer). One exemplary embodiment of a system and programmable processing device is described with reference to FIG. 4 of the above-referenced U.S. application Ser. No. 10/884,638. However, many other embodiments of varying constructions and complexities, both larger and smaller, are also contemplated to be within the scope of the present invention. The system (e.g., concentrator) 20 can comprise, for example, a chamber assembly 22 and a concentration device 28. In a typical embodiment, the chamber assembly 22 may comprise a tissue collection chamber that receives tissue through a conduit 23 and that may be controlled by a control and communication path 24 that is coupled to the programmable processing device 32. Received tissue may be processed, for example, washed, in the chamber assembly 22 and then passed to the concentration device (e.g., processing chamber) 28, which may comprise for example a centrifuge, through another conduit 26.

The concentration device 28 may be operated under control of control and communication path 27 that is coupled to the programmable processing device 32 to produce a composition that, when placed into a patient, may induce at least one of hard and soft tissue formation. The placing into a patient may be achieved using techniques well known in the art. For example, the composition may be suspended in a solution, the solution drawn into a syringe, and the solution injected into the patient from the syringe. In another embodiment, the composition may be applied onto an implant, and the implant may be inserted into the patient.

In typical embodiments, concentrated material (comprising, for example, regenerative cells) passes to an output device, such as, for example, an intervascular delivery device 30, through yet another conduit 29 for, as an example, infusion into a patient. In other embodiments, the concentrated material passes to a secondary device, such as, for example, a bioreactor module 30, through a conduit 29, for further purification or processing before passing to an output device, such as, for example, an intervascular delivery device 30, through a conduit (not shown) for infusion into a patient. The concentrated material can be manually retrieved using, for example, a syringe. In other embodiments, the concentrated material (e.g., a final pellet or other composition comprising regenerative cells) may be automatically moved to a container which may be removed and stored or used as needed. This container may be in any appropriate form or size. For example, the container may even be a syringe. In certain embodiments, the output device may be heat sealed (either automatically or manually) and isolated from the other components of the concentration device for subsequent retrieval and use of the concentrated material in therapeutic applications as described or incorporated by reference herein including re-infusion (e.g., re-implantation) into the patient. The concentrated material may also be subject to further processing as described or incorporated by reference herein either prior to retrieval from the output device or after transfer to, for example, a second system or device.

Operation of the programmable processing device 32 may be partially controlled by input from a user on a user input port 34 that may connect to a user interface panel on the medical device, such as a keypad. An exemplary user interface panel 106 is illustrated as part of an automation device (that may be a programmable processing device) shown in FIG. 4 of U.S. application Ser. No. 10/316,127. In another embodiment, the user input port 34 may comprise an infrared (IR) port that communicates with a remote device such as a programmable digital assistant (PDA). In yet another embodiment, the user input port 34 may comprise one of a wired or wireless interface to an external keypad. Other embodiments of the user input port 34 may comprise a barcode reader, a magnetic strip reader, or a receptacle for a flash card.

The illustrated embodiment of FIG. 1 further comprises a miscellaneous port 40 that may comprise, as examples, a barcode reader, keypad, wireless remote keypad, magnetic strip, or flash card receptacle. According to an exemplary mode of operation, patient data may be entered on the miscellaneous port 40. For example, a patient may wear a wristband having medical information placed thereon in the form of a barcode that enters the miscellaneous port 40 through a barcode reader. In other embodiments, patient information may be coded on a flash card or on a magnetic strip that likewise may be entered into the miscellaneous port 40. The barcode with patient information can be printed on a printer associated with the device and can be manually placed on the output device 30 to ensure delivery of an autologous concentration of material from the device, e.g., autologous regenerative cells.

The programmable processing device 32 may further include a display port 38 capable of communicating with a display device such as a printer or a screen that may be implemented as part of a user interface panel 106 as illustrated, for example, in FIG. 4 of the aforementioned U.S. application Ser. No. 10/316,127. The display port 38 can be used to communicate information regarding managing and servicing the medical device to the user.

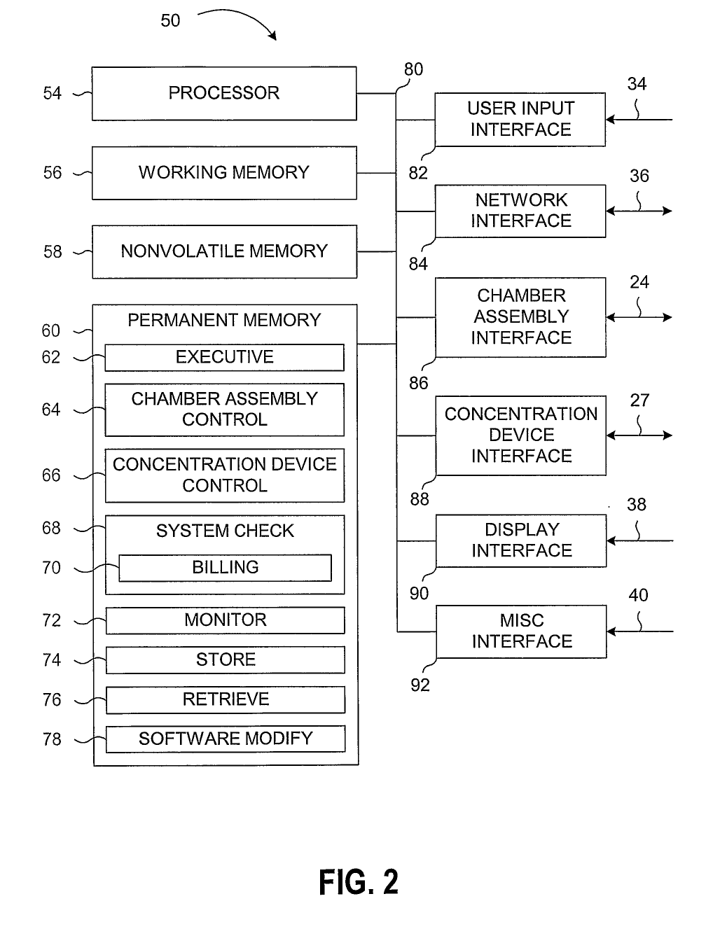

An exemplary embodiment of the programmable processing device 32 is shown in FIG. 2. As illustrated, this embodiment comprises a processor 54 capable of executing instruction sequences. The programmable processing device 32 further comprises working memory 56, permanent memory 60 that stores instruction sequences, a user input interface 82, a chamber assembly interface 86, and a concentration device interface 88. The aforementioned elements can be interconnected by a system bus 80, which enables the processor 54 to communicate with the elements.

This illustrated embodiment of the programmable processing device 32 further comprises a chamber assembly control instruction sequence 64 and a concentration device control instruction sequence 66 that may be stored in permanent memory 60. The processor 54 is able to execute the chamber assembly control and concentration device control instruction sequences 64 and 66, and may thereby control the chamber assembly 22 (FIG. 1) and the concentration device 28 (FIG. 1) by communicating with the chamber assembly interface 86 and the concentration device interface 88 over the system bus 80. The chamber assembly interface 86 is able to communicate with the chamber assembly 22 by means of control and communication path 24 (see also FIG. 1). Likewise, with further reference to FIG. 1, the concentration device interface 88 is able to communicate with the concentration device 28 by means of control and communication path 27.

The embodiment illustrated in FIG. 2 further may comprise a network interface 84, which is capable of communicating with the processor 54 through the system bus 80 and further capable of communicating with an external network over communication path 36. An embodiment of the network interface 84 can comprise a wired interface that may connect, for example, to a network comprising a remote server. Another embodiment of the network interface 84 may comprise a wireless interface having similar capabilities. A modified embodiment may employ a serial interface in addition to or instead of the network interface 84. The serial interface, for example, a Universal Serial Bus (USB) interface, may be capable of communicating with an external device such as a printer. According to yet another embodiment, the programmable processing device can further comprise a display interface 90 capable of communicating with a display device such as a printer or a screen through a display port 38. The programmable processing device 32 further may comprise a miscellaneous interface 92 capable of receiving input from, for example, at least one of a barcode reader, keypad, wireless remote, magnetic strip, or flash card reader.

The permanent memory 60 of the embodiment illustrated in FIG. 2 may have stored therein a system check instruction sequence 68. As an example, the system check instruction sequence 68 may be included in exemplary pre-programmed steps illustrated in FIG. 15B of U.S. application Ser. No. 10/884,638.

Typically, in accordance with embodiments of the present invention, the system check instruction sequence 68 can be executed by the processor 54 once during a normal session of collecting and processing tissue. As such, according to an implementation of the present invention, execution of the system check instruction sequence 68 can provide a means by which a record of use, i.e., a usage event, of the medical device may be created and maintained.

An embodiment of the system check instruction sequence 68 that can be employed in the present invention minimally may cause the processor to receive a charge code, or other charge or identity or use related information or indication (hereinafter “charge code”), from the user input interface 82. The charge code, which may be received on user input port 34, may identify the user and may be associated with, for example, an authorization code, a user rate, and the like that may be used for billing purposes.

Accordingly, in certain implementations of the present invention, execution of the system check instruction sequence 68 minimally can cause the processor 54 to communicate the usage event and the charge code to the network interface 84. The usage event may comprise information concerning, for example, the medical device used, the date, and the time of the usage.

An embodiment of the system check instruction sequence 68 may comprise a billing instruction sequence 70. The billing instruction sequence 70 minimally may cause the processor to generate a bill according to, for example, the user charge code and the usage event. In certain implementations, the billing instruction sequence 70 further minimally may cause the processor to communicate a report to the display interface 90 according to the bill, whence the report may be communicated to the display port 38 for display on a user-readable device such as a printer or screen. Yet another embodiment of the medical device may comprise a display device, for example, a monitor screen, a printer, and the like, the display device being configured to automatically display the report received by the display interface.

Another embodiment of the programmable processing device 32 further may comprise nonvolatile memory 56, for example, flash memory, in which data may be stored that is not lost when power is removed from the programmable processing device 32. The nonvolatile memory 56 may store data such as patient information, user authorization codes, or the like. According to another embodiment, the nonvolatile memory 56 may store instruction sequences, for example, one or more of the instruction sequences shown as being stored in permanent memory 60 in the embodiment illustrated in FIG. 2. Nonvolatile storage of such instruction sequences is advantageously contemplated by the present invention, thereby providing a capability of modifying instruction sequences, for example, upgrading device software, on an as-needed basis. Additionally, nonvolatile memory 56 may store device license information, software patches, operating systems, and the like, allowing such system elements to be upgraded from time to time. A software modify instruction sequence 78 minimally may cause the processor 54 to perform the upgrades just described.

Other embodiments of the programmable processing device 32 further may comprise a monitor instruction sequence 72. The monitor instruction sequence 72 may cause the processor 54 to record or log notable events in nonvolatile memory 56 using a store instruction sequence 74. Notable events may comprise a simple routine use of the medical device, consumption of a given quantity of one or more reagents, use of disposable elements, and the like. A retrieve instruction sequence 76 may cause the processor 54 to retrieve stored data from nonvolatile memory 56 in respond to a command received, for example, from the network interface 84.

Yet another embodiment of the programmable processing device 32 comprises an executive instruction sequence 62 that may be referred to as an operating system in some embodiments. The executive instruction sequence 62 may cause the processor 54 to manage the execution of, for example, at least the aforementioned instruction sequences in order to carry out functions of the medical device.

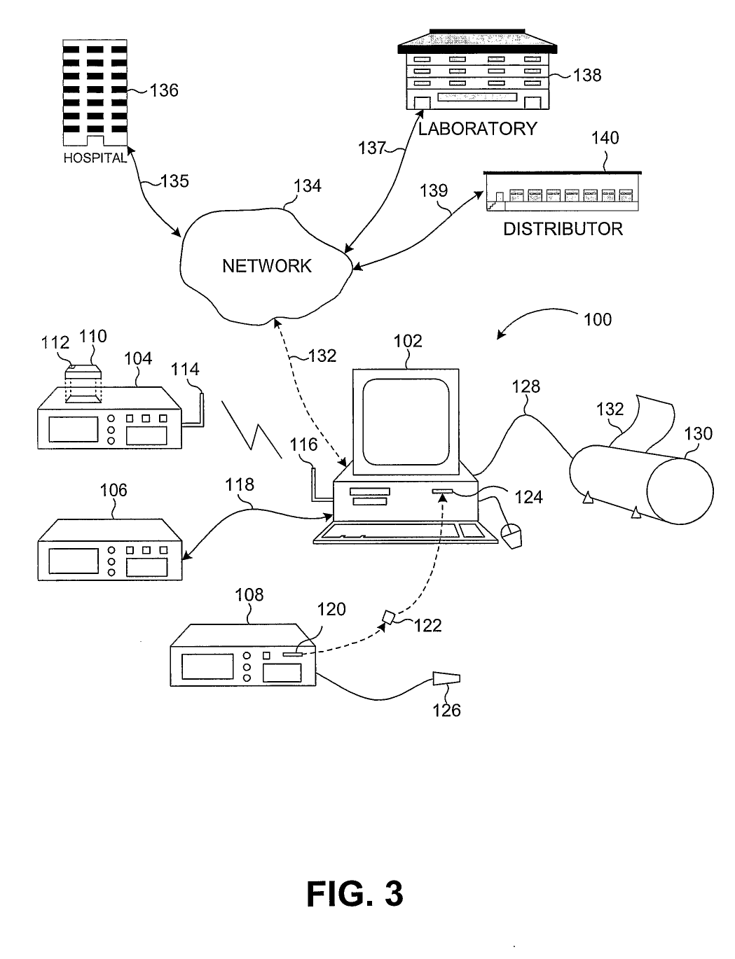

A pictorial diagram of an exemplary embodiment of a medical billing device 100 is depicted in FIG. 3. The medical billing device 100, as illustrated in this figure and in accordance with an aspect the present invention, comprises a server, for example, a personal or laptop computer 102, including a storage device, for example, a hard drive (not shown). The medical billing device 100 further may comprise a printer 130 connected to the personal computer 102 with, for example, a cable 128. The medical billing device 100 may receive information according to usage of at least one medical device. According to an embodiment, the medical billing device 100 may communicate wirelessly to receive usage information, using a wireless network interface 116, with a medical device 104 having a wireless network interface 114 as described above. For example, the network interface 114 may comprise a network interface 84 of a medical device as described in FIGS. 1 and 2. Operation of the medical device 104 may require the use of disposable elements symbolically represented in FIG. 3 by disposables 110. Exemplary embodiments of disposables 110 may comprise a smart chip 112 capable of communicating with a programmable processing device similar to programmable processing device 32 described above with reference to FIGS. 1 and 2. The smart chip or chips 112 may have encoded therein information such as, for example, a lot number and an expiration date for the (e.g., for each of the) disposable or disposables 110.

According to another example, the medical billing device 100 may receive usage information by means of a wired network interface 118 that is coupled to another medical device 106 having a wired network interface as likewise already described. According to yet another example, the medical billing device 100 may receive usage information from a flash memory card 122 that is manually movable from a slot 120 in a medical device 108 to a slot 124 in the medical billing device 100. As a variation on the flash memory card example, an authorization code may be written on the flash memory card 122 by the medical billing device 100. The medical device 108 may in certain implementations be configured to require the presence of a flash memory card 122 in slot 120, and in other implementations further to require that the flash memory card 122 have written thereon a valid authorization code, in order for the medical device 108 to be operable. Another embodiment of the medical device 108 may comprise a barcode reader 126 capable of reading, as examples, data from a patient wrist band, user identification information from an identity badge of a user, and so on.

The medical billing device 100 may maintain a database of information pertaining to, for example, medical devices, charge codes and usage events, on the storage device included in the personal computer 102. Alternatively, part or all of the database, databases, or related information, may be maintained on an external storage medium such as a flash memory card 122.

Referring to FIG. 4, a typical database maintained by, for example, the medical billing device 100, may comprise a user rate table 150. The user rate table 150 may in turn comprise records including a medical device identification field 152, which may contain a medical device identification code, a charge code field 154 and a rate field 156 associated with, for example, a medical device according to, for example, the medical device identification code and a charge code that may occupy the charge code field 154. For example, an entry in the medical device identification field 152 may correspond to one or more of the medical devices 104, 106 and 108 depicted in FIG. 3. The charge code field 154 may comprise identification information for a user such as a physician, a group of users such as a collection of physicians or technicians, an organization such as a hospital or clinic, and the like. Each of these entities may have negotiated with an owner or distributor of various medical devices, for example, at least one of the medical devices illustrated in FIG. 3, a different rate or protocol to be maintained in a rate field 156 according to, for example, the charge code associated with the user.

The medical billing device 100 may receive information from time to time according to usage of one or more medical devices associated with the medical billing device 100. For example, the medical billing device 100 may receive information on the wireless network interface 116 comprising a usage event for the medical device 104 (i.e. an indication that medical device 104 has been used) and a charge code according to the user of the medical device 104.

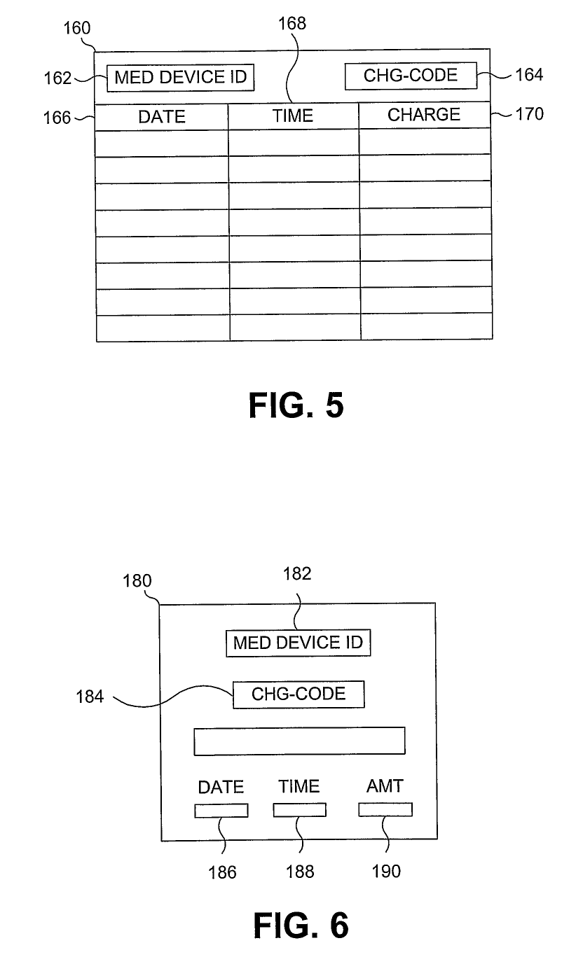

Turning to FIG. 5, a usage table 160, which is stored, for example, within the medical billing device 100, may comprise a medical device identification field 162 and a charge code field 164, and may further comprise records containing date, time, and charge information. A given record in the illustrated example of usage table 160 can be keyed, for example, to a given medical device (e.g., medical device 104) and a given charge code, so that the record can be updated according to pertinent received information.

When usage event information is received, the medical billing device 100 may determine and assign a date and time using methods known in the art. Alternatively, the usage event may include the date and time. The medical billing device 100 may create a record (e.g., in a pertinent usage table 160) and may enter the date into a date field 166 of the record and may enter the time into a time field 168 of the record. The medical billing device 100 further may access user rate table 150 (FIG. 4) to determine a rate according to the charge code in the charge code field 164 and the medical device identification in the medical device identification field 162, and may enter the rate in the charge field 170 of the record. Thus, in the exemplary embodiment, usage table 160 can store information according to the usage of a single medical device by a user associated with a single charge code. In practice, several usage tables similar to usage table 160 may be maintained in the database of (or accessible by) the medical billing device 100, which may service several medical devices and several users.

As another example, the medical billing device 100 may receive information concerning a usage event associated with another medical device, such as medical device 106, the information being received over a wired network connection 118. In another embodiment, usage event information pertaining to yet another medical device, such as medical device 108, can be received from a flash memory card 122 that may be manually transferred from slot 120 in medical device 108 to slot 124 in the medical billing device 100. In these and other instances the medical billing device 100 is able to store or update information in the database according to the received information in a manner similar to that already described.

In yet another example, the medical billing device 100 may receive information regarding remotely located medical devices by means of a network 134. The network 134 may comprise, for example, the Internet, and remotely located medical devices (and/or parts or all of the database) may be located hundreds or thousands of miles away from the medical billing device 100. For instance, the other medical devices may be located in a hospital 136 or a laboratory 138 physically separated from medical billing device 100. In the embodiment illustrated in FIG. 3, medical billing device 102 connects to the network 134 through wired or wireless connection 132. Similarly, the hospital 136 and the laboratory 138 connect with the network 134, respectively, through wired or wireless connections 135 and 137. Information in the database may be updated in such instances using procedures already described.

The medical billing device 100 may, in other embodiments, function as a server or as a peer to peer element capable of communicating with other medical devices as well as medical devices 104, 106, and 108 illustrated in FIG. 3. Medical billing device 100 and other medical devices may cooperate to share information over the network 134, permitting the medical billing device 100 to gather, analyze and present real-time usage data from any desired medical device or devices. The usage data obtained may be useful in marketing medical devices and services, making financial decisions, performing clinical studies, engaging in biomedical research and development, and the like. In some instances the usage data may be sold to a third party.

From time to time, the medical billing device 100 may create a report comprising a bill to be submitted to a user, the bill detailing charges for use of one or more medical devices during a given time period. According to one example, the medical billing device prints a report 132 (FIG. 3) on the printer 130 whenever, for example, a predetermined time elapses or information about a usage event is received. FIG. 6 presents a chart elucidating such a report, which may comprise a form 180, according to a single use of a medical device. The form 180 may in turn comprise a medical device identification field 182 containing medical device identification information, a charge code field 184 containing identification of a user, and fields for date 186, time 188, and amount billed 190. To create the report 132, the medical billing device 100 may access the database, retrieve information from a table similar to usage table 160 shown in FIG. 5, format the information according to the form illustrated in FIG. 6, and print the report.

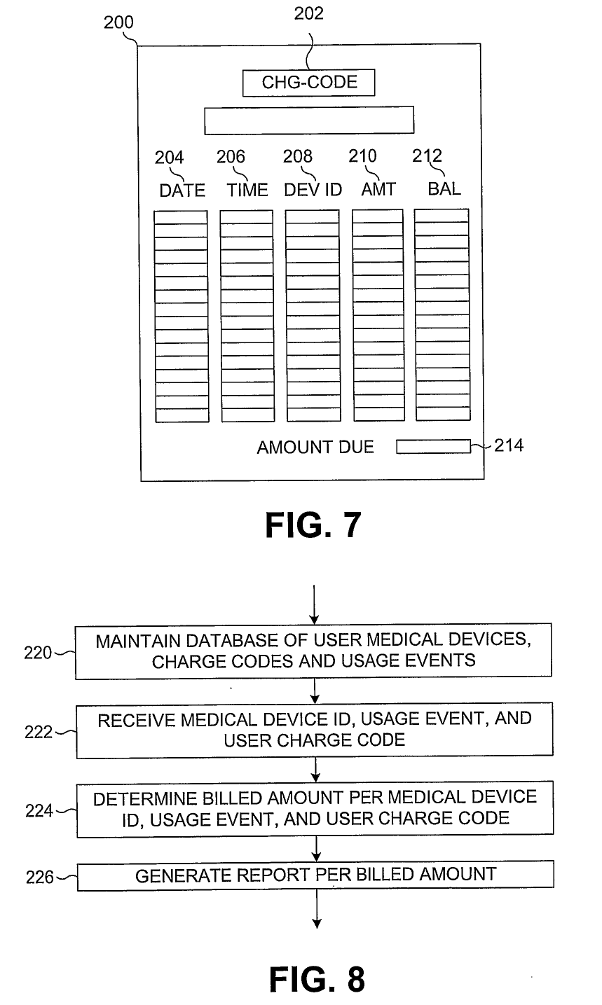

According to one method of operation, the medical billing device 100 may generate billing reports on a periodic basis, such as, for example, daily, weekly, biweekly, monthly, and the like. FIG. 7 is a chart illustrating an exemplary report, which corresponds generally to report 132 (FIG. 3) and which comprises a bill 200 that may be created periodically according to uses of one or more medical devices. To create the bill 200, the medical billing device 100 may access one or more usage tables 160 (FIG. 5) in the database and may retrieve records according to usage of medical devices according to one or more charge codes during a selected time period. The bill 200 then may be created by entering the charge code or codes into a charge code field or fields 202. Further, referring to FIG. 5 as an example, information from the date field 166, the time field 168, the medical device identification field 162, and the charge field 170 may be copied into, respectively, a date field 204, a time field 206, a device identification (ID) field 208, and an amount field 210 of, for example, a line of the bill 200. The bill 200 further may include a running balance field 212 in which amounts from the amount field 210 are accumulated, and a total billed amount may be presented in an amount due field 214.

FIG. 8 is a flow diagram summarizing an implementation of a method of billing a user for use of one or more medical devices. This implementation comprises maintaining a database of medical devices, user charge codes, and usage events at step 220. The database may be maintained on a storage device in a laptop computer, a personal computer, a server, or the like. The database may include tables containing information similar to that depicted in the user rate table 150 shown in FIG. 4 and usage tables similar to usage table 160 illustrated in FIG. 5. A medical device ID, usage event, and user charge code are received at step 222. Various techniques are available for receiving the usage event and associated information. For example, information relating to medical device usage may be received by way of a wired or wireless network connection. In another embodiment, the information may be received on a flash memory card that is manually transferred from a medical device to a medical billing device. According to other embodiments, medical devices may have located thereon or nearby timesheets filled out by operators at times when the medical devices are used. Information from these timesheets may be collected periodically, for example, hourly, daily, or the like, and entered automatically or manually into a medical billing device such as a laptop or personal computer as illustrated in FIG. 3. In modified embodiments, such as may be employed in, for example, a small laboratory, the use of a medical device ID and a user charge code may be obviated. In one such modified embodiment, only a usage event may be received at step 222.

A billed amount per usage event is determined at step 224 according to, for example, the medical device ID and the user charge code received at step 222. According to a representative implementation, the billed amount is determined according to information stored as to the medical device used and the charge code associated with the user of the medical device. A report according to the billed amount is generated at step 226. An implementation of the method can generate a report comprising a bill when, for example, a usage event is received. Another implementation of the method can retrieve stored information pertaining to all medical device usage events by a user associated with a charge code during a selected time period, and a report can be generated according to the total charge accumulated during that time period. A display device, such as a computer monitor, printer, and the like, may display the generated report.

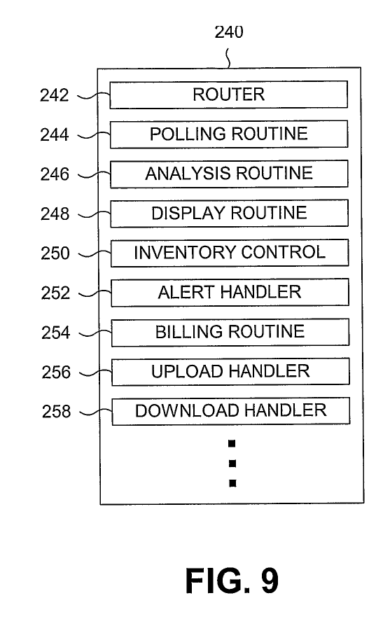

The medical billing device 100 illustrated in FIG. 3 typically comprises at least one processor capable of executing instructions. According to an embodiment, the medical billing device 100 may have stored in computer readable memory, exemplary contents of which are depicted in FIG. 9, instruction sequences 240 comprising a router instruction sequence 242 that minimally may cause the processor to communicate with medical devices, e.g. medical devices 104, 106, and 108, located on a local area network (LAN) and to facilitate communication between designated medical devices connected directly to the network 134 or connected to network 134 through a LAN, wide-area network (WAN), metropolitan area network (MAN), or the like. The instruction sequences 240 further may comprise a poling routine 244 capable of minimally causing the processor to transmit a request to any or all of the aforementioned medical devices for the purpose of retrieving stored information such as usage data as already described. The instruction sequences 240 further may comprise an analysis routine 246 capable of causing the processor to process, e.g., aggregate, organize, and classify, usage data, placing the usage data in a form capable of being readily interpreted by a human observer. A display routine 248, likewise included in the instruction sequences 240, may cause the processor to present the processed usage data on a display device.

The intercommunication capabilities provided by network 134 may permit medical billing device 100 to perform an inventory control function using an inventory control instruction sequence 250 that may be one of the instruction sequences 240. The inventory control instruction sequence 250 may cause the processor to execute an alert handler instruction sequence 252, thereby causing the processor to respond to messages received from medical devices, the messages pertaining, for example, to consumption of disposables and reagents. The inventory control instruction sequence 250 may, in those cases, cause the processor to estimate a need for disposables and reagents, place an order with a manufacturer or distributor 140 (FIG. 3), and/or execute a billing routine 254 that may cause generation of a bill to a customer according to the order. The bill may be delivered electronically, or may, in some embodiments, be printed on paper on-site with the customer, or printed centrally with the billing device 100 and mailed to the customer. Supplies may be automatically shipped to the customer by the manufacturer or distributor, obviating a need for an order to be initiated by the customer.

Another embodiment of the medical billing device 100 (FIG. 3) may comprise an upload handler instruction sequence 256 capable of causing the processor to receive information uploaded from one or more medical devices as described herein. The medical billing device 100 further may comprise a download handler instruction sequence 258 capable of causing the processor to receive information to be downloaded to one or more medical devices that communicate with the network 134. For example, the downloaded information may comprise software upgrades, licenses, software patches, operating systems and the like. An eXecutable Internet may facilitate the upload/download procedures contemplated by the present invention.

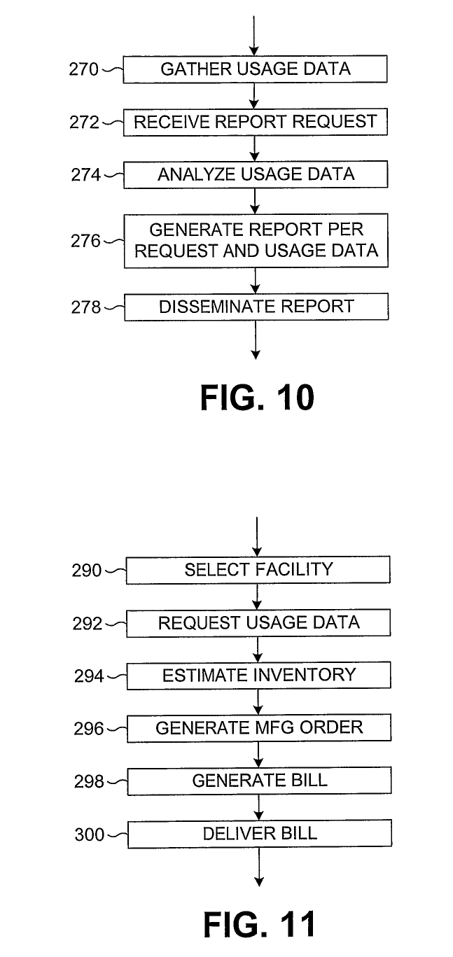

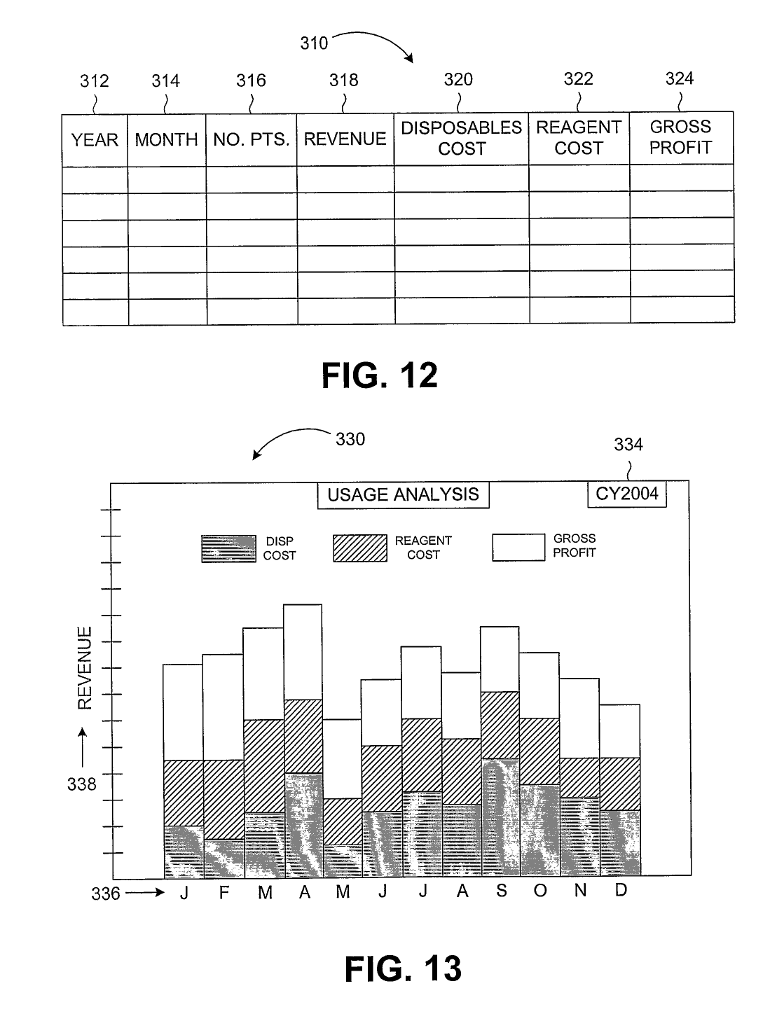

An example of a method of managing usage data according to a collection of medical devices is illustrated in the flow diagram presented in FIG. 10. Usage data is gathered at step 270 using techniques already described. For example, a polling routine 244 described above with reference to FIG. 9 may cause a processor to request usage data from one or more medical devices having access to a network 134 as illustrated in FIG. 3. With usage data available, a report request may be received at step 272. For example, an owner or seller of medical devices may enter a request for a report on a keyboard of the medical billing device 100 (FIG. 3), or another authorized user may request a report from another location having access to the network 134. Typically, the request for a report includes an indication of the type of information desired in the report. See, for example, illustrative reports as described below in FIGS. 12 and 13. The usage data may be analyzed at step 274. The analysis may occur in the medical billing device 100 and may be accomplished by executing analysis routine 246 as described above with reference to FIG. 9. According to the report request and the usage data, a report may be generated at step 276. The report may comprise a graphical presentation in some cases (see, for example, FIG. 13). In other cases, the report may be presented in tabular form as shown in FIG. 12. In either case, the report may be disseminated at step 278.

A variety of reports may be generated according to the method described in FIG. 10. For example, information may be received according to a number of hours of operation of a given medical device since the medical device was initially placed into service. The received information may take the form of usage events as described herein. Alternatively, referring to records maintained in the medical billing device 100, a number of hours of operation of the medical device since routine maintenance or operation was last performed on the medical device may be determined. By comparing such numbers of hours with historical data regarding the operation of similar medical devices, repair personnel can be dispatched remotely to perform maintenance on the medical device, while likelihood of device malfunction is low, in time to prevent loss of revenue due to unscheduled downtime for repairs or failure of the medical device. Service costs can be minimized as a result of repair personnel being sent out with the right parts a first time, and only when needed.

In other instances, the report request received at step 272 (FIG. 10) may comprise an alert from a medical device, letting a user and/or a manufacturer or distributor know when there is a problem with the medical device or, for example, when reagents or disposables are running low.

Some users of medical devices may choose to use disposables manufactured by a third party, rather than disposables manufactured by a manufacturer of the medical devices. Using intelligent methods as described herein, a medical device may be capable of recognizing such events and of generating an alert that can be received by the medical billing device 100 (FIG. 3). According to an embodiment of the present invention, user fees may be adjusted upward when third-party disposables are used, thereby reducing an incentive for users to deviate from recommended practices.

According to another embodiment, medical devices interconnected as described herein are required to communicate with, e.g., the medical billing device 100 (FIG. 3) from time to time. If a given medical device fails to communicate with the medical billing device 100 for longer than a prescribed time interval, e.g. one hour, then the medical device may be presumed to be stolen and/or cease to function in whole or in part.

Other reports generated by the method described in FIG. 10 may comprise historical data regarding, for example, usage of a class of medical devices by all or a subpopulation of users. This historical data may be made available (e.g., for a fee) to users and/or prospective users so that, for example, physicians can optimize usage parameters according to patient profiles or to assess risks.

FIG. 11 is a flow diagram illustrating another example of how usage data may be managed according to the present invention. A facility is first selected at step 290. The facility may comprise a hospital 136 (FIG. 3), a laboratory 138, or other facility having access to network 134. Usage data may be requested at step 292, for example, by causing a processor in medical billing device 100 to execute a polling routine 244 as described above with reference to FIG. 9. When usage data is received, inventory may be estimated at step 294. An inventory control instruction sequence 250 may cause a processor in the medical billing device 100 (FIG. 3) to estimate inventory in the selected facility, taking into account, for example, all relevant medical devices that are in use at that facility. According to the estimated inventory, a manufacturing order may be generated at step 296. The manufacturing order may be generated by the processor in the medical billing device 100 (cf. FIG. 3) according to the inventory control instruction sequence 250 (FIG. 9). The manufacturing order may be communicated to the network 134 through connection 132 and received from the network 134 by a distributor 140 (FIG. 3) through connection 139. The distributor 140 then may fill the order, shipping products directly to the selected facility. A bill may be generated at step 298 in accordance with supplies requested in the manufacturing order at step 296, and the bill may be delivered to the facility at step 300. According to a typical embodiment, the bill can be communicated electronically through the network 134 as described herein.

FIG. 12 is a chart illustrating an example of a tabular report of financial aspects of operation of one or more medical devices. The report shown comprises a table 310 having a field 312 for a year of operation, e.g. CY 2003, FY 2004, or the like. A month field 314 may identify twelve months in each of twelve rows in the table 310. A number of patients field 316 may contain a number of patients treated with the one or more medical devices during each month listed. The number of patients information may be received as described herein by polling medical devices able to communicate with the network 134 (FIG. 3). A revenue field 318 may contain revenue generated by the medical devices during each month. Similarly, a disposables cost field 320 and a reagent cost field 322 may comprise costs of, respectively, disposables and reagents during each month, the disposables and reagents costs likewise being received by, for example, polling the medical devices. An analysis routine 246 (FIG. 9) may cause a processor in medical device 100 to calculate a gross profit 324 (revenue minus disposables cost minus reagent cost) for the collection of medical devices for each month listed in the table 310.

Information presented in table 310 of FIG. 12 may alternatively be presented graphically as depicted in FIG. 13. In the illustrated example, disposables cost (gray), reagent cost (cross hatched) and gross profit (white) are presented in a stacked bar-graph 330, whereby the height of each bar represents total revenue 338 received from operation of medical devices during each of twelve months 336 in CY 2004 334.

As disclosed in U.S. Application No. 60/338,856 with reference to FIG. 4, an embodiment of a medical device may have integrated therein a printer component comprising a hardware printer and software driver. Alternatively, the device may comprise a software printer driver and an external interface, for example, a USB interface, capable of communicating with an external printer. Another embodiment of a medical device further may include a storage device, for example, flash memory, and permanent memory having stored therein instruction sequences capable of causing a processor to provide billing functions. The embodiment thereby may provide billing functions pertaining to or following the method described in FIG. 8 in accordance with, for example, the particular medical device. For instance, a table similar to that illustrated in FIG. 4 may be stored in flash memory, and the medical device may cause printing of a report that is similar to that described in connection with FIG. 6 after each use of the medical device or periodically as described herein.

In view of the foregoing, it will be understood by those skilled in the art that the methods of the present invention can facilitate monitoring, managing and servicing medical devices, and in particular billing for use of medical devices for separating and concentrating clinically safe regenerative cells from adipose tissue. The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

1. An automated medical system comprising:
a chamber assembly configured to receive adipose tissue removed from a patient, disaggregate said adipose tissue, and separate adipose-derived regenerative cells from said adipose tissue;
a concentration device comprising a filter, elutriator, or centrifuge connected to said chamber assembly and configured to concentrate said adipose-derived regenerative cells;
a programmable processing device configured to communicate with and control said chamber assembly and said concentration device, wherein the programmable processing device records a notable event of said automated medical system.

2-38. (canceled)

39. The automated medical system according to claim 1, wherein the notable event comprises a recordation of the consumption of a reagent.

40. The automated medical system according to claim 1, wherein the notable event comprises a recordation of the consumption of a disposable element.

41. The automated medical system according to claim 1, wherein the notable event comprises routine use of the medical device.

42. The automated medical system according to claim 1, wherein the notable event comprises a recordation of patient information.

43. The automated medical system according to claim 1, wherein the notable event comprises a recordation of billing information.

44. The automated medical system according to claim 1, further comprising a conduit connected to the chamber assembly, wherein said conduit is configured to receive adipose tissue.

45. The automated medical system according to claim 1, further comprising an output chamber connected to the concentration device.

46. The automated medical system according to claim 1, wherein the programmable processing device comprises a user input port.

47. The automated medical system according to claim 1, wherein the programmable processing device comprises a display port.

48. The automated medical system of claim 1, further comprising a server in communication with said automated medical system, wherein the server is configured to receive a history of usage of said automated medical system and maintain a database of said history of usage.

49. The automated medical system of claim 48, wherein the server is in communication with the medical system through a recording device that can be accessed by the medical system and the server.

50. The automated medical system according to claim 49, wherein the recording device is a flash memory.

51. The automated medical system according to claim 48, wherein the server is in communication with the medical system through a network.

52. The automated medical system according to claim 51, wherein the network is wireless.

53. The automated medical system according to claim 48, wherein the network is wired.

54. The automated medical system according to claim 48, further comprising a second medical device that is in communication with the server.

55. The automated medical system according to claim 48, further comprising a history of items consumed by the medical system.

56. The automated medical system according to claim 55, wherein the items consumed are disposable elements.

57. The automated medical system according to claim 55, wherein the items consumed are reagents.

58. A method for recording a history of usage of an automated medical system that is configured to isolate adipose-derived regenerative cells, comprising:
introducing adipose tissue removed from a patient into the automated medical system of claim 1;
processing said adipose tissue removed from said patient to isolate said adipose-derived regenerative cells; and
recording the usage of said automated medical system.

59. The method of claim 58, wherein said automated medical system further comprises a server in communication with said medical system, wherein the server is configured to receive a history of usage of said automated medical system and maintain a database of said history of usage.

60. A method for billing a user for the isolation of adipose-derived regenerative cells comprising:
providing the automated medical system of claim 1;
recording the usage of said automated medical system;
receiving a usage history from the automated medical system;
determining a billed amount according to the usage history; and
generating a report of the billed amount.

61. The method according to claim 60, wherein a report is generated when a usage event is received.

62. The method according to claim 60, further comprising receiving a charge code.

63. A method of maintaining an inventory of items consumed by an automated medical system comprising;
providing the automated medical system of claim 1;
maintaining a database of an inventory of consumable items;
receiving a history of usage of the automated medical system; and
recalculating the inventory of consumable items.

64. The method according to claim 63 further comprising generating a report of the inventory of consumable items.

65. The method according to claim 63 further comprising receiving a history of consumable items that are added to the inventory of consumable items.

66. The method according to claim 63, further comprising generating an alert when the inventory of consumable items is below a predetermined amount.

67. The method according to claim 63, further comprising ordering more consumable items from a vendor when an inventory of consumable items is below a predetermined amount.

68. The method of claim 63, wherein the inventory of consumable items comprises an inventory of disposable elements.

69. The method of claim 63, wherein the inventory of consumable items comprises an inventory of reagents.

70. A method of maintenance of an automated medical system, comprising:
providing the automated medical system of claim 1;
maintaining a database of the maintenance history of said medical system;
receiving the maintenance history from the medical system; and
determining if maintenance is needed.

71. The method according to claim 70, further comprising providing a communication to elicit maintenance of said device.

72. The method according to claim 70, wherein maintenance is needed if the time of operation since the last maintenance is greater than a predetermined amount.

73. The method according to claim 70, wherein maintenance is needed if a recent history of usage deviates from a recorded historical usage by a predetermined amount.
